(19) United States
(12) Patent Application Publication
(10) Pub. No.: US 2025/0115868 A1
(43) Pub. Date: Apr. 10, 2025

(54) SEQUENTIAL APPLICATION OF MACROPHAGES FOR WOUND HEALING

(71) Applicant: Drexel University, Philadelphia, PA (US)

(72) Inventors: Kara Lorraine Spiller, Philadelphia, PA (US); Sina Nassiri, Philadelphia, PA (US)

(21) Appl. No.: 18/737,396

(22) Filed: Jun. 7, 2024

Related U.S. Application Data
(63) Continuation of application No. 15/767,569, filed on Apr. 11, 2018, now abandoned.
(60) Provisional application No. 62/242,580, filed on Oct. 16, 2015.

(51) Int. Cl. C12N 5/0786; A61K 9/00; A61K 39/00; A61P 17/02

(52) U.S. Cl. CPC C12N 5/0645; A61K 40/17; A61K 40/24; A61K 40/40; A61P 17/02; A61K 9/0014; A61K 9/0019

(57) ABSTRACT

The application relates to the healing of wounds. Provided herein are methods directed to treatment of hard-to-heal or chronic wounds by sequential administration of M1 and M2 macrophages to the wound site

### CROSS-REFERENCE TO RELATED APPLICATIONS

This application is a continuation of U.S. patent application Ser. No. 15/767,569, filed Apr. 11, 2018, which is a national stage of International Patent Application No. PCT/US2016/057092, filed Oct. 14, 2016, which claims the benefit of the priority of U.S. Provisional Patent Application No. 62/242,580, filed Oct. 16, 2015 (expired), each of which are incorporated herein by reference.

### STATEMENT REGARDING FEDERALLY SPONSORED RESEARCH OR DEVELOPMENT

This invention was made with government support under Contract No. 1R01HL130037-01 awarded by the National Institutes of Health, National Heart, Lung, and Blood Institute. The government has certain rights in the invention.

### BACKGROUND OF THE INVENTION

Macrophages are known to be important regulators of wound healing. In normal wound healing, macrophages exhibit a classically activated, pro-inflammatory “M1” phenotype in the early stages of wound healing (1-4 days), and at later stages (4-14 days), the macrophage population shifts to an “M2” phenotype. M1 macrophages are often believed to be detrimental to healing, while M2 macrophages are believed to promote healing. This good-vs.-evil M1-M2 paradigm is believed by many in the biomaterials, regenerative medicine, and wound healing communities because chronic wounds with impaired healing also have persistently elevated levels of M1 macrophages. Indeed, the detrimental effect of M1 macrophages has been described in numerous injury situations, including diabetic ulcers [1, 2], chronic venous ulcers [3], spinal cord injury [4], atherosclerotic lesions [5], traumatic spinal cord injury [4], inflammatory renal disease [6], and biomaterial implantation [7, 8].

Wound healing remains a challenge in clinical medicine.

### SUMMARY OF THE INVENTION

This application is directed toward the treatment of wounds. The application provides methods for the treatment of a wound by sequential administration of exogenous M1 and M2 macrophages. Other aspects and advantages of the present invention are described further in the following detailed description of the preferred embodiments thereof.

### BRIEF DESCRIPTION OF THE DRAWINGS

FIGS. 1A-1D reflect day 10 images of the effects of sequential M1 and M2a activation on angiogenesis in vitro as described in Example 4. Lining in each image reflects construct vascularization, which lining appears in red in the original imaging. FIG. 1A shows scaffold vascularization in a control of Human Adipose Microvascular Endothelial Cells (HAMEC) expressing tdTomato on porous gelatin scaffold together with human adipose-derived Mesenchymal Stem Cells (MSC). FIG. 1B shows scaffold vascularization by sequential seeding of macrophages activated with IFNγ on day 3 and macrophages activated with TL4+IL13 on day 6. FIG. 1C shows scaffold vascularization by seeding of macrophages activated with IFNγ+ LPS on day 3. FIG. 1D shows scaffold vascularization by seeding of macrophages activated with TL4+IL13 on day 6. Scale bar=250 μm; n=3.

FIGS. 2A-2B provide a cluster analysis of publicly available data set using validated M2c genes. FIG. 2A shows a soft clustering of top 100 M1, 100 M2a and 17 M2c genes in human wound healing using publicly available data [17]. FIG. 2B shows the composition of the genes in each cluster, calculated by dividing the number of phenotype-associated genes in each cluster by the total number of genes for that phenotype in all four clusters.

### DETAILED DESCRIPTION OF THE INVENTION

A purpose of the embodiments described herein is to treat wounds, including surgical wounds and chronic wounds. M1 macrophages are known to be associated with early stages of the normal wound healing process, with the macrophage population shifting to an M2 phenotype at later stages of healing. Chronic wounds have persistent or elevated levels of M1 macrophages. The failure to transition from M1 to M2 macrophages in diabetic ulcers, for example, may be due to defective (M1) macrophages, impairing efferocytosis, and thus inhibiting a natural signal to switch from M1 to M2. However, M2 macrophages applied without prior application of M1 macrophages impair healing. Applying exogenously stimulated M1 and M2 cells in appropriate succession promotes wound healing in sites where the natural healing process is ineffective.

M1 macrophages stimulate angiogenesis, a required part of wound healing. M2 macrophages act at later stages to support the early actions of M1 macrophages. Exogenous addition of M2 macrophages to a wound without prior application of M1 macrophages impairs healing. While chronic wounds have been associated with elevated levels of M1 macrophages relative to healthy or acute wounds, elevating levels of M1 macrophages promotes healing of chronic diabetic ulcers, provided that this is followed by elevated levels of M2 macrophages. In some embodiments, delivery of exogenously activated M1 and M2 macrophages to wounds supplements the natural healing process. In other embodiments, delivery of exogenously activated M1 and M2 macrophages to wounds provides macrophages as substitutes for macrophages that are defective.

Macrophages are differentiated from monocytes. Upon tissue damage or infection, macrophages are recruited to the affected site. Macrophages are polarized in response to the tissue and environment to which they are exposed. M1 macrophages can inhibit cell proliferation and cause tissue damage. These macrophages are associated with Th1 responses. For example, LPS and interferon gamma drive polarization to the M1 phenotype. M1 macrophages are also referred to as classically activated macrophages. These macrophages can be further subdivided into M1a and M1b macrophages based on their stimulation and/or expression. Unless otherwise provided, any embodiment referencing M1 macrophages encompasses embodiments directed to M1a and/or M1b macrophages.

M2 macrophages promote cell proliferation and tissue repair. These macrophages are associated with Th2 responses. For example, IL-4 (IL4) drives polarization to the M2 phenotype, specifically M2a polarization. M2 macrophages are also referred to as alternatively activated macrophages. They are subdivided into M2a, M2b, M2c, and M2d macrophages based on their stimulation and/or expression. For example, IL-10 drives M2c polarization, and VEGF drives M2d polarization. Unless otherwise provided, any embodiment referencing M2 macrophages encompasses embodiments directed to M2a and/or M2b and/or M2c macrophages. In further embodiments referencing M2 macrophages, the embodiments are directed to M2a and/or M2c macrophages.

At the site of a wound in a healthy subject, the immune response passages from M1 to M2 expression, with each successive stage leading from inflammation to resolution of inflammation to tissue repair to normal cell or tissue function. While biomaterial control over the phenotype of endogenously recruited macrophages may be sufficient to promote healing in some situations, especially in healthy people, the macrophages in hard-to-heal or chronic wounds, e.g., diabetic ulcers, are defective and/or and have impaired ability to clear the wound of debris and transition from M1 into the M2 phenotype(s). Thus, the exogenous application of macrophages to the wound site is beneficial. Further, unexpectedly, in certain embodiments, administration of M1 macrophages followed by M2 macrophages to a wound is advantageous. In certain embodiments, administration of M1 macrophages without subsequent administration of M2 macrophages is effective in treatment of a wound. In another embodiment, it would be advantageous to administer M2 macrophages following a few days of inflammatory stimulus, e.g., 1, 2, 3, 4, 5, 6, 7, 8, 9, 10, 11, 12, 13, or 14 days, or any combination of hours, minutes, and seconds between 1 and 14 days. The embodiments described herein enable the supplementation, enhancement, and correction of the wound healing process in a subject.

As used herein, the singular form of a term is intended to encompass the plural, and vice versa, unless otherwise noted.

All ranges referred to herein include all sub-ranges, integers, and fractions of integers, unless otherwise provided.

The terms “comprising,” “comprises,” “contains,” “containing,” “has,” “have,” “having,” “include,” includes,” “including”, and the like, are used interchangeably and indicate that the subject is open ended, unless otherwise noted.

The terms “consist,” “consists,” “consisting,” and the like, are used interchangeably and indicate that the subject is open ended, unless otherwise noted.

Throughout this application, where compositions, components, methods, or steps are described as required in one or more embodiments, additional embodiments are contemplated and are disclosed hereby for fewer compositions, components, methods, or steps, and for fewer compositions, components, methods, or steps in addition to other compositions, components, methods, or steps. All compositions, components, methods, or steps provided herein may be combined with one or more of any of the other compositions, components, methods, or steps provided herein unless otherwise indicated.

The term “autologous” in reference to cells or tissue, unless otherwise noted, is intended to mean that the cell or tissue is obtained, directly or indirectly, from the same individual subject to which it is to be delivered. Unless otherwise noted, the term “autologous” includes cells or tissues derived from cells or tissues obtained, directly or in indirectly, from the same individual subject to which it is to be delivered.

The term “allogeneic” in reference to cells or tissue, unless otherwise noted, is intended to mean that the cell or tissue is obtained, directly or indirectly, from a different individual of the same species than the subject to which it is to be delivered. Unless otherwise noted, the term “allogeneic” includes cells or tissues derived from cells or tissues obtained, directly or in indirectly, from a different individual of the same species than the subject to which it is to be delivered.

The term “exogenous,” unless otherwise noted, refers to anything grown, prepared, polarized, cultured, or modified outside of the subject.

The terms “subject” and “patient” are, unless otherwise noted, used interchangeably to refer to the target or recipient of a treatment or composition described herein. These terms include, unless otherwise specified, all vertebrates, including all mammals, including humans. Unless otherwise noted, an embodiment using the term “subject” and “patient” is intended to include an embodiment directed solely to solely to mammals, solely to humans, solely to non-human mammals, solely to companion mammals, solely to companion vertebrates, solely to companion mammals, solely to non-human animals, and solely to non-human mammals.

The term “wound” is intended to refer to an injury to living tissue of a subject. Unless otherwise noted, embodiments referring to a wound include embodiments where the wound is one in which the skin is cut or broken, including surgical wounds. Further, embodiments referring to a wound include embodiments where the epidermis is broken. Still further, embodiments referring to a wound include embodiments where the dermis is cut or broken. In still other embodiments referring to a wound, the wound is a tissue other than of the skin, including internal surgical wounds. Unless otherwise noted, embodiments referring to a wound include embodiments where the wound is an ulcer. Still further, a wound may also be a burn. The burn may be of the third degree.

The term “acute wound” refers to a wound which heals consistent with the timing or process conventional to the type and severity of the wound for the species of the subject.

The term “chronic wound” refers to a wound which does not heal consistent with the timing or process conventional to the type and severity of the wound for the species of the subject.

The terms “hard-to-heal” or “refractory” refer to a wound which does not heal using conventional therapies available as of the filing date of this application.

The term “diabetic wound” refers to any wound in an individual having diabetes.

The terms “disease”, “disorder”, or “condition” are used herein to refer to any manifestations, symptoms, or combination of manifestations or symptoms, recognized or diagnosed as connected with a chronic wound, hard-to-heal wound, or diabetic wound.

The terms “treat,” “treating,” “treatment,” and the like, as used herein, refer to any method or composition used to reduce, improve, alleviate, ameliorate, or reduce the severity of, a wound or condition as defined herein.

The term “wound composition” refers to a composition that may be applied to a wound to promote healing or prevent further injury.

The term “pharmaceutically acceptable carrier” or “diluent” is intended to include any and all solvents, dispersion media, coatings, antibacterial and antifungal agents, isotonic and absorption delaying agents, adjuvants and the like, compatible with administration to humans. In one embodiment, the diluent is saline or buffered saline.

The term “effective amount,” unless otherwise noted, means an amount which provides a therapeutic benefit to a subject.

In one embodiment, a method of treating a wound of a subject is provided comprising the sequential administration of exogenous M1 macrophages and exogenous M2 macrophages to the wound. In a further embodiment, the exogenous M1 macrophages delivered are M1a, M1b, or M1a and M1b macrophages. The exogenous M2 macrophages delivered are one or more of M2a, M2b, M2c, and M2d macrophages. In a further embodiment, the exogenous M1 macrophages delivered are one or more of M2a, M2b, and M2c. In still a further embodiment the exogenous macrophages are M2a and M2c.

By sequential administration, the M2 macrophages may be delivered to a subject from 1 to 12 months, 1 to 2 months, 1 to 4 weeks, 1 to 2 weeks, 2 to 14 days, 2 to 7 days, or 3 to 4 days, inclusive, following administration of the M1 macrophages. In further embodiments, the M2 macrophages may be delivered to a subject 2, 3, 4, 5, 6, 7, 8, 9, 10, 11, 12, 13, or 14 days, or 1 week, 2 weeks, 3 weeks, 4 weeks, 1 month, 2 months, 3 months, 4 months, 5 months, 6 months, 7 months, 8 months, 9 months, 10 months, 11 months, or 12 months following administration of the M1 macrophages. In still further embodiments, the M2 macrophages may be delivered to a subject in any combination of months, days, hours, minutes, and seconds within these ranges. For example, the M2 macrophages may be delivered to a subject 2 days, 6 hours, following administration of the M2 macrophages.

In further embodiments, the administration of M1 macrophages is sequenced or repeated prior to administration of M2 macrophages. For example, M1b macrophages may be administered 1, 3, 4, 5, 6, 7, 8, 9, 10, 11, 12, 13, or 14 days following administration of M1a macrophages. In still further embodiments, the M1b macrophages may be delivered to a subject in any combination of hours, minutes, and seconds from 1 to 13 days, or any range or sub-range thereof, following administration of M1a macrophages. Instead or, or in addition to, any of the above sequencing, administration of M1a and/or M1b may be repeated 1, 2, 3, 4, 5, or more times, from 1, 3, 4, 5, 6, 7, 8, 9, 10, 11, 12, 13, or 14 days from first administration, and spaced at any combination of days, hours, minutes, and seconds from 1 to 13 days, or any range or sub-range thereof.

Likewise, in embodiments, the administration of M2 macrophages is sequenced or repeated. For example, M2b macrophages may be administered 1, 3, 4, 5, 6, 7, 8, 9, 10, 11, 12, 13, or 14 days following administration of M2a macrophages. M2c macrophages may be administered 1, 3, 4, 5, 6, 7, 8, 9, 10, 11, 12, 13, or 14 days, or 1 week, 2 weeks, 3 weeks, 4 weeks, 1 month, 2 months, 3 months, 4 months, 5 months, 6 months, 7 months, 8 months, 9 months, 10 months, 11 months, or 12 months following administration of M2a macrophages, or following administration of M2b macrophages. In still further embodiments, the M2b and/or M2c macrophages may be delivered to a subject in any combination of months, weeks, days, hours, minutes, and seconds from 1 to 12 months, 1 to 4 weeks, or 1 to 13 days, or any range or sub-range within these periods, following administration of other M2 macrophages. Instead or, or in addition to, any of the above sequencing, administration of M2a, M2b and/or M2c may be repeated 1, 2, 3, 4, 5, or more times, from 1, 3, 4, 5, 6, 7, 8, 9, 10, 11, 12, 13, or 14 days, or 1 week, 2 weeks, 3 weeks, 4 weeks, 1 month, 2 months, 3 months, 4 months, 5 months, 6 months, 7 months, 8 months, 9 months, 10 months, 11 months, or 12 months, from first administration of an M2 macrophage, and spaced at any combination of months, weeks, days, hours, minutes, and seconds from 1 to 12 months, 1 to 4 weeks, or 1 to 13 days, or any range or sub-range thereof.

In embodiments, M2a macrophages may be administered 1, 3, 4, 5, 6, 7, 8, 9, 10, 11, 12, 13, or 14 days following administration of M2b macrophages. M2a macrophages may be administered 1, 3, 4, 5, 6, 7, 8, 9, 10, 11, 12, 13, or 14 days, or 1 week, 2 weeks, 3 weeks, 4 weeks, 1 month, 2 months, 3 months, 4 months, 5 months, 6 months, 7 months, 8 months, 9 months, 10 months, 11 months, or 12 months following administration of M2c macrophages, or following administration of M2b macrophages. In still further embodiments, the M2a and/or M2b macrophages may be delivered to a subject in any combination of months, weeks, days, hours, minutes, and seconds from 1 to 12 months, 1 to 4 weeks, or 1 to 13 days, or any range or sub-range within these periods, following administration of other M2 macrophages. Instead or, or in addition to, any of the above sequencing, administration of M2c, M2a and/or M2b may be repeated 1, 2, 3, 4, 5, or more times, from 1, 3, 4, 5, 6, 7, 8, 9, 10, 11, 12, 13, or 14 days, or 1 week, 2 weeks, 3 weeks, 4 weeks, 1 month, 2 months, 3 months, 4 months, 5 months, 6 months, 7 months, 8 months, 9 months, 10 months, 11 months, or 12 months, from first administration of an M2 macrophage, and spaced at any combination of months, weeks, days, hours, minutes, and seconds from 1 to 12 months, 1 to 4 weeks, or 1 to 13 days, or any range or sub-range thereof.

In embodiments, M2c macrophages may be administered prior to M2a macrophages, without prior or subsequent administration of M1 macrophages.

In another embodiment, a method of treating a wound of a subject is provided comprising administering exogenous M0 (relatively unactivated) or M1 macrophages to the wound. In a further embodiment, the macrophages are M0 macrophages. In another embodiment, the macrophages are M1 macrophages. In a further embodiment, the M1 macrophages delivered are M1a, M1b, or M1a and M1b. In still a further embodiment, M0 macrophages may be delivered before, after, or concurrently with M1 macrophages.

When M0 macrophages are delivered before or after M1 macrophages (including M1a and/or M1b macrophages), the sequential delivery to a subject of M0 and M1, or M1 and M0, may be spaced from shortly after administration of the first macrophages through 14 days afterward. Administration of M0 or M1 may be 1 minute to 14 days, 2 to 7 days, or 3 to 4 days, inclusive, following administration of the M1 or M0 macrophages, respectively. In further embodiments, the second macrophages of M1 or M0 may be delivered to a subject immediately following first administration through 1, 2, 3, 4, 5, 6, 7, 8, 9, 10, 11, 12, 13, or 14 days following first administration. In still further embodiments, the M0 or M1 macrophages may be delivered to a subject in any combination of hours, minutes, and seconds within these ranges. For example, the M0 macrophages may be delivered to a subject 2 days, 6 hours, following administration of the M1 macrophages. Likewise, the M1 macrophages may be delivered to a subject 2 days, 6 hours, following administration of the M0 macrophages.

In further embodiments, the administration of M0 or M1 macrophages is repeated prior to administration of M1 or M0 macrophages, respectively. For example, administration of M0 and/or M1 may be repeated 1, 2, 3, 4, 5, or more times, from 1, 3, 4, 5, 6, 7, 8, 9, 10, 11, 12, 13, or 14 days from first administration, and spaced at any combination of days, hours, minutes, and seconds from 1 minute to 13 days, or any range or sub-range thereof.

In still further embodiments, single or repeated administration of M0 or M1 macrophages may follow single or repeated administration of M2 macrophages.

Still further provided are uses of M0, M1, and/or M2 macrophages, M0, M1a, M1b, M2a, M2b, M2c, and M2d macrophages as described herein in the treatment of a wound. Still further provided are uses of the macrophages in the preparation of medicaments useful in the treatment of a wound. The medicaments are useful in methods for wound treatment, comprising sequential administration of M0, M1a, M1b, M2a, M2b, M2c, and M2d macrophages as described herein. Still further provided is the use of one of more of the macrophages described herein to treat a wound. The treatment may comprise any of the methods described herein.

The methods described herein may be used for any type of wound. In specific embodiments, the wound may be a chronic wound, an acute wound, an open wound, a closed wound, a clean wound, a contaminated wound, an infected wound, a diabetic wound, an ulcer, a burn wound, a diabetic ulcer, a foot sore, or a skin sore. However, the embodiments described herein are not so limited.

The wound may be acute. The wound may be chronic, hard-to-heal, or refractory. The wound may also be associated with a metabolic disease or other disease in which natural healing is diminished. The wound may be a diabetic wound. The wound may be a diabetic ulcer. The wound may be a wound of a patient with Type I or Type II diabetes.

The subject or patient may be a mammal, or more specifically a human. In further embodiments, still any subject or patient as defined or otherwise described herein may be treated according to the invention. Any composition described herein may be formulated for one or more species of subject or groups of subjects.

Macrophages of the phenotypes described above, including M0, M1a, M1b, M2a, M2b, M2c, and M2d macrophages, may be isolated from an animal, such as a mammal, such as a human, for delivery according to the methods above. In other embodiments, the macrophages to be delivered may be prepared from monocytes.

Monocytes or macrophages may be isolated from sources known to one of skill in the art, including from human peripheral blood, or enriched leukocyte fractions of human peripheral blood, and used in the embodiments described herein. In one embodiment, monocytes may be obtained from any source exogenous to the subject. Macrophages may be obtained from culture with macrophage colony stimulating factor (MCSF). Monocytes may be polarized into M1, including M1a, M1b, and M2, including M2a, M2b, M2c, and M2d, macrophages using polarizing factors known to one of skill in the art. For polarization to M1, interferon gamma and/or lipopolysaccharide (LPS) and/or TNF (tumor necrosis factor)-alpha may be used. In a further embodiment, interferon gamma is used. In still a further embodiment, interferon gamma and LPS are used. For polarization to M2a, IL (interleukin)-4 or IL-13 (IL13) may be used, either separately or in combination. IL13 may be used for polarization to M2. For polarization to M2b, immune complexes, LPS, or glucocorticoids may be used, either separately or in any combination. For M2c, IL-10, TGF-beta, or glutocorticoids may be used, either separately or in any combination. For M2d, IL-6, leukocyte inhibitory factor, macrophage chemotactic factor, or VEGF. Still other factors known in the art may be used to polarize macrophages, and the factors used are not intended to limit this application.

Exogenous macrophages used herein may also be prepared by any method known to one of skill in the art, including but not limited to the methods described elsewhere [14].

The exogenous macrophages obtained by any method described herein or known in the art may be delivered to a subject according to the methods described herein by any means. These macrophages may be administered alone, as pharmaceutical compositions in combination with diluents and/or carriers and/or buffers and/or other components. In one embodiment, they may be administered in saline. In another embodiment, in a hydrogel. Among other formulations, both saline and hydrogel formulations are contemplated for delivery by injection. Other components may include cytokines, cells, or other agents conventionally used to promote wound treatment or healing. Compositions may include stabilizers, antioxidants, and/or preservatives. Compositions may include, e.g., neutral buffered saline or phosphate buffered saline.

Carriers may include pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. Carriers also includes any and all coatings, antibacterial and antifungal agents, and absorption delaying agents, and the like that are compatible with the activity of the components, e.g., cells, to be delivered, and are physiologically acceptable to the patient. Supplementary active compounds may also be incorporated into the compositions. Pharmaceutically acceptable salt of the compound or molecule useful within the invention.

Other ingredients that may be included are excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Still other additional ingredients that may be included in the pharmaceutical compositions used in the practice of the invention and are known in the art and described elsewhere [15].

Effective amounts of the macrophages and other aspects of a pharmaceutical composition may be determined by one of skill in the art, including by a physician. Such amounts may be determined by with consideration of the age and/or weight of a patient, and further by the size, condition, location, and/or severity of the wound or wounds to be treated. In one embodiment, administration of from 1 to 100 million macrophages is appropriate. Further ranges/subranges, and integers within those ranges/subranges, are also contemplated, including but not limited to from 1 to 10 million, 1 to 5 million, 1 to 1 million, 1 to 500,000, 1 to 400,000, 1 to 300,000, 1 to 250,000, 1 to 200,000, 1 to 150,000, 1 to 100,000, 1 to 50,000, 1 to 40,000, 1 to 30,000, 1 to 20,000, 1 to 10,000, 1 to 5,000, 1 to 2,500, 1 to 1,000, 1 to 100, 10 million to 100 million, 50 million to 100 million, 75 million to 100 million.

In one embodiment, the macrophages are administered at least topically to the site of the wound. In other embodiments, the macrophages are administered at least transdermally to the site of the wound. The macrophages may also be administered at least via injection to the site of the wound, i.e., the wound site. In one embodiment, the macrophages may be administered in saline. In another embodiment, the macrophages may also be administered in a hydrogel. The wound site may include the area of damaged tissue and/or tissue peripheral to the wound, e.g., uninjured skin. In certain embodiments, one, two, three, four, five, or more injection site(s) may be used within or adjacent the wound. Where adjacent the wound, the injection site(s) may be 1 mm through 10 mm from the wound, including, 1, 2, 3, 4, 5, 6, 7, 8, 9, or 10 mm from the wound, and fractional increments thereof.

Still other methods encompass the methods described herein in addition to conventional therapies, including but not limited to the application of oxygen topically.

Also provided herein is the use of exogenous M1 macrophages and exogenous M2 macrophages to treat a wound, or the use in a regimen comprising sequential delivery of the M1 macrophages and M2 macrophages, consistent with the methods described herein. Further provided is the use of exogenous M1 macrophages and exogenous M2 macrophages in the preparation of a medicament useful in a wound treatment regimen comprising sequential delivery of the M1 macrophages and M2 macrophages consistent with the methods described herein.

Provided herein is the use of exogenous M1 macrophages and exogenous M2 macrophages to treat a wound of a subject. Also provided is the use of exogenous M1 macrophages and exogenous M2 macrophages in a regimen to treat a wound of a subject. The regimens may comprise administering to said subject said M2 macrophages following said M1 macrophages, or may comprise administering to said subject said M2 macrophages prior to said M1 macrophages. Each administration may be divided into at least two sequential administrations. Further embodiments include use of exogenous M2a and M2c macrophages in a regimen, in either order, and with or without prior administration of M1 macrophages.

Further provided is the use of exogenous M1 macrophages and exogenous M2 macrophages in the preparation of a medicament useful in a wound treatment regimen comprising sequential delivery of the M1 macrophages and M2 macrophages. The regimens may comprise administering to said subject said M2 macrophages following said M1 macrophages, or administering to said subject said M2 macrophages prior to said M1 macrophages. Each administration may be divided into at least two sequential administrations. Further embodiments include medicaments useful in wound treatment regimens utilizing exogenous M2a and M2c macrophages, for administration in either order, and with or without prior administration of M1 macrophages.

Still further provided are kits comprising one of more of the macrophages described herein in one or more vials, tubes, or other suitable vessels. The kit may further comprise a syringe or other medical instrument suitable to deliver a composition to a subject.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The embodiments described above further include that matter contained within the following examples, the claims, and any other component of the application.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein. The specific embodiments described in the Examples are intended to be embodiments of the invention.

### Example 1—Isolation and Culture of Primary Human Macrophages

Monocytes are isolated from enriched leukocyte fractions of human peripheral blood using sequential Ficoll and Percoll density gradient centrifugations. Monocytes are cultured at 37° C. and 5% CO₂ in ultra low attachment flasks for 5 day at a density of 0.4×10⁶ cells/cm² and 1.0×10⁶ cells/ml of complete media (RPMI media supplemented with 10% heat-inactivated human serum, 1% penicillinstreptomycin, and 20 ng/ml macrophage colony stimulating factor (MCSF)). Macrophages are polarized over the next 1-6 days by culturing at 1.0×10⁶ cells/ml in complete media with 100 ng/ml IFN-gamma and 100 ng/ml lipopolysaccharide (LPS, Sigma Aldrich) for M1 or 40 ng/ml IL4 and 20 ng/ml IL13 for M2, with a media change at day 3. At the media change, the media of another group of M1 macrophages is switched to M2-polarizing media and the media of a group of M2 macrophages is switched to M1-polarizing stimuli, in order to characterize the ability of macrophages to switch phenotypes. Unactivated macrophages were also cultured over the same time periods (M0), resulting in three groups through day 3 (M0, M1, M2) and five groups between days 4 and 6 (M0, M1, M2, M1/M2, M2/M1).

### Example 2—Characterization of Macrophage Phenotype

At days 1, 2, 3, 4, and 6, macrophages are collected by gentle scraping and centrifugation. The number of viable cells are determined at each time point by trypan blue exclusion. Macrophages from each time point are characterized for expression of known M1 and M2 markers by quantitative RT-PCR. For flow cytometry, cells are dual-stained with APC-conjugated CCR7 and FITC-conjugated CD206. Corresponding isotype controls are used as recommended by the manufacturer. Labeled cells are analyzed using a FACSCalibur flow cytometer and the CellQuest software. Data is processed using FlowJo software (Treestar).

To determine the proportion of cells staining for a given marker at high or low levels, the mean intensity of staining of the M0 population is used as a threshold.

In other words, cells staining more intensely for CCR7 than the mean of the M0 population are considered CCR7^{hi}, while those staining less intensely than the mean of the M0 population are considered CCR7^{lo}. This analysis is performed similarly for CD206^{hi} and CD206^{lo} populations, allowing determination of the proportion of cells that are both CCR7^{hi} and CD206^{lo} and those that are both CCR7^{lo} and CD206^{hi}.

At each time point, the supernatant is frozen at −80° C. until analysis by enzyme-linked immunosorbent assays (ELISA). Secreted M1 markers include tumor necrosis factor-alpha (TNF-alpha) and VEGF (Peprotech) and M2 markers include CCL18 (R&D Systems) and PDGF-BB (Peprotech).

### Example 3—Mouse Wound Healing Model Protocol

1. Genetically diabetic male 12-week-old mice (db/db;Lepr^{db}) and their heterozygous male littermates (db^{+/-}) are taken.
2. Four animals are housed per cage pre-surgery and alone post-surgery, and are maintained in an animal care facility with a 12-hour light/dark cycle throughout the acclimation (4 weeks) and test periods.
3. The animals are 16 weeks old at the time of surgical wounding.
4. All survival surgery is done using aseptic technique:
   a. Sterile surgical gloves;
   b. Steam autoclaved instruments; and
   c. Preservation of sterile field using sterilized drape material or disposable pads.
5. The mice are anesthetized by Isofluorane.
6. Mice have the wound region of the back shaved and all hair removed with a commercially available hair depilatory cream. The sites are surgically prepared with betadine (or other appropriate surgical prep solution i.e. Nolvasan) followed by alcohol.
7. The surgical field is maintained at all times through aseptic technique. All animals are placed on a warming pad covered with a sterile drape to maintain body temperature throughout the surgical procedure.
8. After the surgical preparation, the wound positions are marked using sterile surgical marking pen (i.e. Accu-line surgical marking pen, or similar surgical pen).
9. Two wounds, one on each side of the midline, are made on the shaved dorsum of the mice using a sterile 6-8 mm punch biopsy tool (single use, disposable).
10. Full-thickness wounds extending through the panniculus carnosus are excised (if needed, an Iris scissor is used to cut any attached tissue).
11. To help avoid contraction of the underlying panniculosus carnosus and promote wound repair mainly through granulation tissue formation and re-epithelialization similar to human skin repair, a 12-mm-diameter donut-shaped splint made of a 0.5-mm-thick silicone sheeting is affixed to the skin using an immediate-bonding adhesive (Krazy Glue or Vetbond) and interrupted 6-0 nylon sutures. If required, semi-occlusive dressing (Tegaderm) is then be applied to cover the wound.

### Example 4—Contribution of Macrophage Phenotype to In Vitro Tissue Vascularization Methods

Human adipose microvascular endothelial cells expressing tdTomato together with human adipose-derived mesenchymal stem cells (MSC) were pre-seeded on porous gelatin scaffolds (Gelfoam, Pfizer) to generate self-assembled vascular networks that can be used to investigate vascularization dynamics in vitro via live imaging. THP-1 monocyte-derived macrophages were activated for 48 h using either IFNγ+ LPS to generate pro-inflammatory (M1) macrophages, or IL4+IL13 to generate IL4-stimulated macrophages, gently scraped and added to the 3-dimensional model of tissue vascularization on days 3 and 6, respectively, during vessel growth. The effects of macrophage phenotype on network development in vitro were monitored over time using a Zeiss LSM 700 confocal microscope; images were analyzed using Fiji, Matlab, Angiotool and Imaris software in terms of network complexity, vessel area, length and diameter, and number of branches.

### Results

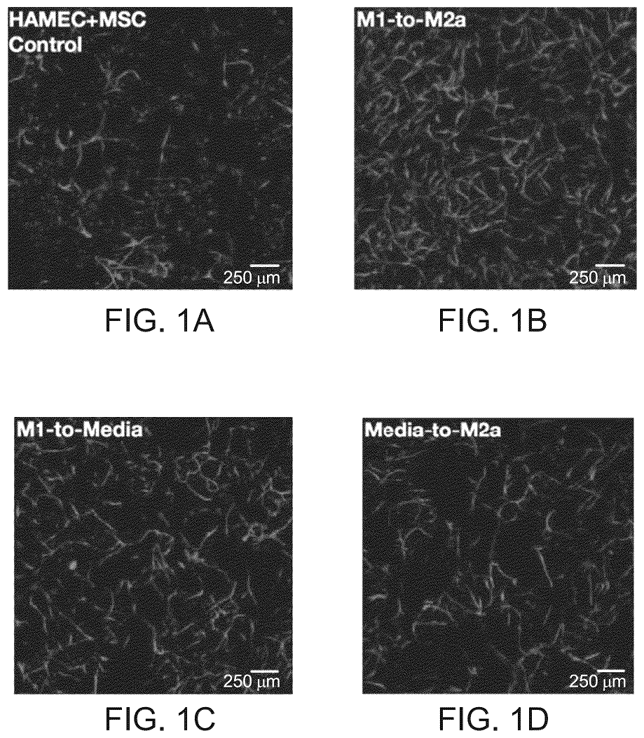

Image analysis revealed that pro-inflammatory and IL4-stimulated macrophages both induced changes in the formation of tissue vasculature in vitro (FIGS. 1A-1D). Most notably, while the individual phenotypes enhanced network complexity relative to control constructs without macrophages, sequential activation of pro-inflammatory and IL4-stimulated macrophages caused vast construct vascularization and promoted vessel elongation.

### Example 5—Longitudinal Analysis of Gene Expression Trends in Human Would Healing Methods

As a preliminary analysis of macrophage activation during human wound healing, publicly available data from microarray studies characterizing heterogeneous wound tissue were used because no studies have been conducted where macrophages were first isolated from human wounds and then profiled at the whole genome level. Three publicly available data sets of gene expression at various time points pre- and post-injury were identified using the Gene Expression Omnibus (GEO) and European Bioinformatics Institute (EBI) repositories [17-19]. Only one of the three datasets, that produced by Gabriel et al. [17], was determined to contain data of sufficient quality for further analysis, as the others either contained significant batch effects [18] or low numbers of replicates and time points [19]. The selected data set was a burn wound model of healthy human skin undergoing aesthetic scarification, taking tissue biopsies immediately before injury, and then 1 hour, 6 days, 25 days, 60 days and 80 days post-injury to analyze changes in gene expression over the course of wound healing. Raw intensity data from Illumina HumanWG-6 v3.0 Expression BeadChips were downloaded from the European Bioinformatics Institute (EBI), and pre-processing was performed with the lumi package. Specifically, expression values were obtained by background correction, a variance-stabilizing and log base 2 transformation, and quantile normalization. QC checks were performed with replicate correlation and pairwise MA plots. Sample-specific quality weights were also quantified using limma's arrayWeights function and all samples were included in downstream analysis. Probe identifiers were annotated with the lumiHumanIDMapping file, and duplicate HUGO gene symbols were combined via a maximum mean method. In total, 48802 probes were mapped to 19344 unique gene symbols.

The top 100 enriched genes for the M1 and M2a phenotypes in terms of fold change over M0, determined from Method 3 of RNASeq data analysis discussed above, and all 17 validated M2c genes were used to create a 217 gene expression matrix from the wound healing dataset. Soft clustering was then performed with the mFuzz algorithm, which creates global clustering structures, to determine groups of genes that were expressed similarly over time [20, 21]. The optimal number of clusters was determined to be four as this resulted in the greatest number of highly correlated genes or “core genes” in each cluster. Since genes are able to contribute to more than one cluster, a correlation coefficient threshold of 0.6 was used to identify the core genes in each cluster and the “fuzzifier” value, which prevents clustering of random data, was estimated via the Schwaemmle and Jensen relation [22]. The relative proportions of macrophage phenotype-associated genes in each cluster were then calculated by dividing the number of genes in the cluster by the total number of genes that mapped to any cluster for each phenotype.

### Results

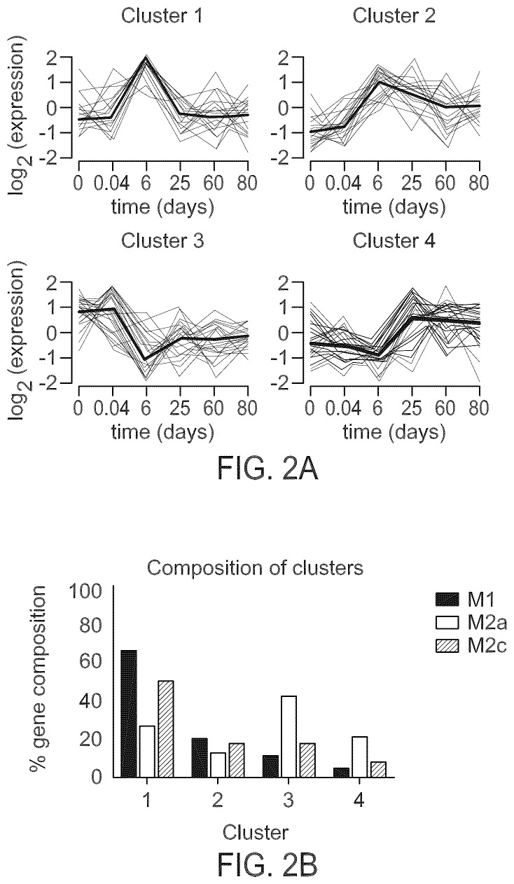

In a preliminary analysis to identify the timing of M2c macrophage-related gene expression in human wound healing, we analyzed a publicly available data set of wound tissue following burn injury [17] for expression over time of the top ˜100 M1 and M2a genes identified using RNA-seq as well as the 17 validated M2c genes. Of the 217 genes analyzed, 126 genes were assigned to at least one of four clusters, which emerged based on similarities in gene expression trends over time. Cluster 1 was composed of genes that increased at day 6 before rapidly returning to baseline (FIG. 2A). This cluster contained over 60% of the mapped M1- and M2c-related genes, and only 20% of M2a-related genes (FIG. 2B). Cluster 2 contained genes that peaked at day 6 and gradually declined thereafter (FIG. 2A). The composition of genes in this cluster was approximately equal for M1-, M2a-, and M2c-related genes (FIG. 2B). Cluster 3 contained genes that were primarily downregulated by day 6 following injury and then returned to baseline thereafter (FIG. 2A.) The composition of this cluster was primarily M2a-related genes (FIG. 2B). Finally, Cluster 4 contained genes that were upregulated at day 25 after injury (FIG. 2A). M2a-related genes were the primary contributor to this cluster (FIG. 2B).

### DISCUSSION

Using next generation sequencing and subsequent validation by Nanostring, we identified 39 genes that were upregulated in the M2c phenotype relative to unactivated (M0) macrophages, and only 17 genes that were differentially upregulated in the M2c phenotype compared to M0, M1 and M2a macrophages. The identified M2c markers are involved in biological pathways necessary during the initiation of healing, and were found to be upregulated at early time points following injury in a human wound healing data set. M2c macrophages may play a role in the early stages of wound healing, in contrast to the characterization of “M2” macrophages as cells that act at later stages. With the identification of genetic markers specific for the M2c phenotype, future studies should focus on the role of these cells in vitro and in vivo.

### REFERENCES

1. Mirza, R E., et al., Sustained inflammasome activity in macrophages impairs wound healing in type 2 diabetic humans and mice. Diabetes, 2014. 63(3): p. 1103-14.
2. Mirza, R. E., et al., Blocking interleukin-1beta induces a healing-associated wound macrophage phenotype and improves healing in type 2 diabetes. Diabetes, 2013. 62(7): p. 2579-87.
3. Sindrilaru, A., et al., An unrestrained proinflammatory M1 macrophage population induced by iron impairs wound healing in humans and mice. J Clin Invest, 2011. 121(3): p. 985-97.
4. Kigerl, K. A., et al., Identification of two distinct macrophage subsets with divergent effects causing either neurotoxicity or regeneration in the injured mouse spinal cord. J Neurosci, 2009. 29(43): p. 13435-44.
5. Khallou-Laschet, J., et al., Macrophage plasticity in experimental atherosclerosis. PLoS One, 2010. 5(1): p. e8852.
6. Wang, Y., et al., Ex vivo programmed macrophages ameliorate experimental chronic inflammatory renal disease. Kidney Int, 2007. 72(3): p. 290-9.
7. Brown, B. N., et al., Macrophage phenotype as a predictor of constructive remodeling following the implantation of biologically derived surgical mesh materials. Acta Biomater, 2012. 8(3): p. 978-87.
8. Brown, B. N., et al., Macrophage phenotype and remodeling outcomes in response to biologic scaffolds with and without a cellular component. Biomaterials, 2009. 30(8): p. 1482-91.
9. Spiller, K. L., et al., The role of macrophage phenotype in vascularization of tissue engineering scaffolds. Biomaterials, 2014. 35(15): p. 4477-88.
10. Jetten, N., et al., Wound administration of M2-polarized macrophages does not improve murine cutaneous healing responses. PLoS One, 2014. 9(7): p. e102994.
11. Spiller, K. L., et al., Sequential delivery of immunomodulatory cytokines to facilitate the M1-to-M2 transition of macrophages and enhance vascularization of bone scaffolds. Biomaterials, 2015. 37: p. 194-207.
12. Nassiri, S., et al., Relative Expression of Pro-Inflammatory and Anti-Inflammatory Genes Reveals Differences Between Healing and Nonhealing Human Chronic Diabetic Foot Ulcers. J Invest Dermatol, 2015.
13. Khanna, S., et al., Macrophage dysfunction impairs resolution of inflammation in the wounds of diabetic mice. PLoS One, 2010. 5(3): p. e9539.
14. U.S. Patent Application Publication No. US2015/0147300.
15. Remington's Pharmaceutical Sciences (1985, Genaro, ed., Mack Publishing Co., Easton, PA.
16. International Patent Application No. WO 2015/077401.
17. Gabriel, V. A., E. A. McClellan, and R. H. Scheuermann, Response of human skin to esthetic scarification. Burns, 2014. 40(7): p. 1338-44.
18. Bronneke, S., et al., Genome-wide expression analysis of wounded skin reveals novel genes involved in angiogenesis. Angiogenesis, 2015. 18(3): p. 361-71.
19. Greco, J. A., 3rd, et al., A microarray analysis of temporal gene expression profiles in thermally injured human skin. Burns, 2010. 36(2): p. 192-204.
20. Futschik, M. E. and B. Carlisle, Noise-robust soft clustering of gene expression time-course data. Journal of bioinformatics and computational biology, 2005. 3(04): p. 965-988.
21. Kumar, L. and M. E. Futschik, Mfuzz: a software package for soft clustering of microarray data. Bioinformation, 2007. 2(1): p. 5-7.
22. Schwammle, V. and O. N. Jensen, A simple and fast method to determine the parameters for fuzzy c-means cluster analysis. Bioinformatics, 2010. 26(22): p. 2841-2848.

Any document (including but not limited to any patent, patent application, publication, and website) listed herein is hereby incorporated herein by reference in its entirety. While these developments have been disclosed with reference to specific embodiments, it is apparent that other embodiments and variations of this invention are devised by others skilled in the art without departing from the true spirit and scope of the developments. The appended claims include such embodiments and variations thereof.

1.-32. (canceled)

33. A method of treating a wound of a subject comprising the sequential administration of exogenous M1 macrophages and exogenous M2 macrophages to said wound, wherein said M1 macrophages are administered to said wound, and said M2 macrophages are administered at least 1 day and not more than 2 days following administration of the M1 macrophages.

34. The method of claim 33, wherein said M2 macrophages are administered at least 2 days following administration of said M1 macrophages.

35. The method of claim 33, wherein said M2 macrophages are administered at least 1 day and not more than 2 days following administration of said M1 macrophages.

36. The method of claim 33, wherein said administration of exogenous M2 macrophages comprises administration of M2a macrophages followed by administration of M2c macrophages and said M2c macrophages are administered at least 1 week and not more than 3 weeks following administration of said M2a macrophages.

37. The method of claim 33, wherein said administration of exogenous M2 macrophages comprises administration of M2a macrophages followed by administration of M2c macrophages and said M2c macrophages are administered at least 2 weeks and not more than 3 weeks following administration of said M2a macrophages.

38. The method according to claim 33, wherein said wound is refractory.

39. The method according to claim 33, wherein said wound is a chronic wound.

40. The method according to claim 33, wherein said wound is a diabetic ulcer.

41. The method according to claim 33, wherein
(a) said administration of exogenous M1 macrophages is divided into at least two sequential administrations;
(b) said administration of exogenous M2 macrophages is divided into at least two sequential administrations;
(c) said administration of exogenous M2 macrophages comprises administration of M2a macrophages followed by administration of M2c macrophages; or
(d) said administration of exogenous M2 macrophages comprises administration of M2c macrophages followed by administration of M2a macrophages.

42. The method according to claim 33, wherein said M1 macrophages and said M2 macrophages are independently autologous or allogeneic.

43. The method according to claim 33, wherein said M2 macrophages are M2a macrophages.

44. The method according to claim 33, wherein said M2 macrophages are M2c macrophages.

44. The method according to claim 33, wherein said subject is a mammal.

46. The method according to claim 33, wherein said subject is a human.

47. The method according to claim 33, wherein said M1 macrophages are administered topically or via injection to the site of the wound.

48. The method according to claim 33, wherein said M2 macrophages are administered topically or via injection to the site of the wound.

49. The method according to claim 33, wherein said administration of at least one of said M2 macrophages or said M1 macrophages is divided into at least two sequential administrations.
